(19) United States
(12) Patent Application Publication
(10) Pub. No.: US 2011/0189109 A1
(43) Pub. Date: Aug. 4, 2011

(54) COMPOSITIONS COMPRISING FENUGREEK HYDROCOLLOIDS

(76) Inventors: Pratibha Sudhir Pilgaonkar, Mumbai (IN); Maharukh Tehmasp Rustomjee, Mumbai (IN); Anikumar Surendrakumar Gandhi, Mumbai (IN); Rupali Kedar Suvarnapathaki, Mumbai (IN)

(21) Appl. No.: 13/122,337

(22) PCT Filed: Oct. 1, 2009
(86) PCT No.: PCT/IN09/00544

§ 371 Date: Apr. 1, 2011

(30) Foreign Application Priority Data
Oct. 3, 2008 (IN) 2123/MUM/2008

(51) Int. Cl. A61K 8/73; A61Q 11/00; A61Q 19/00; C11D 3/60; C09K 3/00; A23G 9/42; A23L 1/187; A23L 1/36

(52) U.S. Cl. 424/49; 514/777; 510/513; 510/238; 252/194; 426/565; 426/579; 426/629

(57) ABSTRACT

The present invention discloses compositions comprising fenugreek hydrocolloids comprising soluble and insoluble dietary fibers. The present invention further relates to the use of these compositions comprising fenugreek hydrocolloids as healthcare, personal care, food, household care and industrial products.

### FIELD OF THE INVENTION

The present invention relates to compositions comprising fenugreek hydrocolloids comprising soluble and insoluble dietary fibers. The present invention also relates to the use of these compositions comprising fenugreek hydrocolloids as healthcare, personal care, food, household care and industrial products.

### BACKGROUND OF THE INVENTION

Hydrocolloids, often called gums, are hydrophilic polymers, of vegetable, animal, microbial or synthetic origin. They increase the viscosity of water by either binding water molecules (hydration) or by absorbing and encapsulating the water into their interwoven macromolecules, at the same time restricting the mobility of the water. Hydrocolloids are used in a variety of industrial sectors to perform a number of functions including thickening and gelling aqueous solutions, stabilizing foams, emulsions and dispersions, inhibiting ice and sugar crystal formation and controlled release of flavors etc. The commonly used hydrocolloids of natural origin are, for example, agar agar, carrageen, tragacanth, gum arabic, alginates, pectin, guar gum, carob bean gum, starch, dextrins and gelatin.

Various uses of hydrocolloids or gums in healthcare, personal care or food industry have been disclosed. Japanese Patent Application JP2005013099A2 teaches a frozen dessert containing 0.25-3 wt. % of a stabilizer, comprising galactomannan, preferably locust bean gum, and tamarind seed polysaccharides in a ratio of (1:9)-(9:1). Japanese Patent Application JP2007006732A2 discloses frozen confectionery getting into gel state when thawed containing glucomannan, galactomannan and tamarind seed gum as a freezing stabilizer. The galactomannan comprises locust bean gum and also carrageenan. U.S. Pat. No. 5,066,508 highlights essentially homogeneous and storage-stable liquid chocolate milk products, formulated from milk bases including a suspension of cocoa particulates and a suspension stabilizing amount of a xanthan gum/galactomannan admixture adapted for the formation of an aqueous gel therefrom, wherein the ratio by weight of xanthan/galactomannan ranges from 80/20 to 20/80. U.S. Patent Application 20050075497 relates to making galactomannan hydrocolloids for use in, for example, food and fodder composition, personal care product like shampoos, hair colorants, comprising swelling split of tamarind, fenugreek, cassia, locust bean, tara or guar with water and wet-mincing to give substantially pure hydrocolloids and derivatives thereof and using said hydrocolloids as gelling and thickening agents. European Patent Application EP1104652A1 discloses preparation of hydrocolloid confectionery product, for example, gum, jelly or pastille involving mixing hydrocolloid(s) (hydrocolloid is selected from the following agarose, gellan, pectin and/or carrageenan) and remaining ingredients, cooking formulation mixture, shaping cooked mass, and triggering cooked mass. U.S. Pat. No. 5,336,515 discloses polysaccharide hydrocolloid containing food products like frozen desserts or mayonnaise. Herein a reduced fat or fat free food product having a moisture content above 45% by weight is provided wherein the reduced fat level is effected by substituting an aqueous dispersion for fat, said dispersion containing polysaccharide hydrocolloid, insoluble fiber and protein at a weight ratio of from 1:0.2-3.5:1.1-4.5. PCT Application WO0007715A1 discloses hydrocolloidal compositions recovered from the liquid fraction obtained by subjecting oat or barley substrates to a heat-shearing treatment that are rich in soluble fiber, principally beta-glucan, and are substantially free of insoluble fiber particles. Dispersions of these compositions are smooth in texture and are useful as texturizers and nutritional substitutes for dairy products in food compositions.

U.S. Patent Application 20070140992A1 discloses oral care composition, for example, toothpaste, mouthwash, gel, toothpowder, edible film comprising a hydrocolloid selected from a plant extract and/or seaweed extract used for taste masking of essential oils comprising menthol, eucalyptol, methyl salicylate or thymol. European Patent 1381282 relates to palatable compositions comprising a plant seed oil selected from a ribes fruit, evening primrose or borage plant, a hydrocolloid base, a sweetening agent and an aqueous phase wherein the hydrocolloid base consists of starch and at least one thickening agent like xanthan gum or alternatively two or more thickening agents selected from the group consisting of guar gum, carageenan, cellulose derivatives and alginic acid and salts thereof.

French Patent Application FR2883473A1 discloses topical cosmetic composition, useful to induce the tanning of the skin, to fight against cutaneous aging and to protect skin from UV rays, comprises an extract of chicle gum and a vehicle. PCT Application WO06081996A1 teaches hydrophilic gel system, useful for cosmetic and skin care applications, comprises a detachable carrier foil and hydrogel containing specific amount of karaya gum and less content of water. U.S. Pat. No. 6,403,066 discloses the use, in cosmetic compositions intended for a temporary shaping of keratin fibres, of guar gum as the sole shaping and/or shape-retention agent for the keratin fibres, in particular the hair, the eyelashes or the eyebrows.

Fenugreek has been used as folk medicine since ancient days. Typically the major constituents of fenugreek seeds have been identified as proteins 20-25%, dietary fiber 45-50% having mucilaginous soluble fiber 15-30% and insoluble fiber also 15-30%, fixed fatty acids and essential oils 6-8% and steroidal saponins 2-5%.

The galactomannan fraction of fenugreek endosperm, which increases the viscosity when dissolved in water, is often referred to as ‘gum’. The major function of galactomannans in the plant is to enable the endosperm to imbibe a large amount of water during seed hydration and, thus, protect the germinating seed from subsequent drought stress. Galactomannan is a polysaccharide made of galactose combined with mannan, high molecular compound of mannose. Fenugreek galactomannan has galactose and mannose present in a ratio of 1:1. Fenugreek galactomannan has an emulsifying property that mixes water and oil, increases viscosity and forms aqueous colloids. Fenugreek galactomannan may have potential use as a medicinal natural food additive for lowering the level of sugar and cholesterol in the blood.

European Patent Application EP0742007A1 discloses the use of Indian senna and fenugreek galactomannan(s), in cosmetic or pharmaceutical products for topical use, and have moisturizing, softening, immunomodulatory and immunostimulatory effects. U.S. Pat. No. 5,847,109 teaches galactomannan emulsions and comestible products containing the same wherein galactomannan isolated from fenugreek has been used as a slow-release cosmetic carrier for lipid(s) and steroid(s), to regulate bowel movements and to reduce post-prandial insulin, cholesterol and glucose levels. PCT Application WO0174371A1 discloses fenugreek mucilages and galactomannans comprising high thickening power, useful as thickeners, stabilizers or soluble fibers in food, cosmetic or pharmaceutical applications. Further, Japanese Patent Applications JP2004203799A2, JP2004203800A2, JP2004203803A2 disclose usage of cationized fenugreek gum for the development of personal care compositions.

The above mentioned prior art discloses the use of hydrocolloids or gums including fenugreek in various industries. There are, however, limitations arising due to the use of only soluble dietary fibers such as galactomannans or gums as hydrocolloids in the different compositions for such applications which may include stickiness, lower emulsifying properties, etc. Through rigorous experimentation the present inventors have surprisingly found that the presence of insoluble dietary fibers along with soluble fibers as hydrocolloids in different compositions helps reduce the stickiness, improve the mouth feel or texture of the products for food, pharmaceutical or personal care. Further, it has also been found that hydrocolloids from fenugreek employed in the compositions of the present invention provide better stabilizing, water holding and emulsifying properties due to the synergistic presence of soluble and insoluble dietary fibers. Further, it was unexpectedly found that rather than utilizing gums or soluble dietary fibers and insoluble dietary fibers from different fiber sources for synergistic benefits, it is simpler, easier and more effective to use fenugreek hydrocolloids comprising both soluble and insoluble dietary fibers obtained from fenugreek in compositions for use in healthcare, personal care, food, household care and industrial products.

### OBJECTS OF THE INVENTION

It is an object of the present invention to provide compositions comprising fenugreek hydrocolloid comprising soluble and insoluble dietary fiber.

It is another object of the present invention to provide compositions comprising fenugreek hydrocolloid comprising soluble and insoluble dietary fiber for use as healthcare, personal care, food, household care and industrial products.

It is another object of the present invention to provide compositions comprising fenugreek hydrocolloid comprising soluble and insoluble dietary fiber wherein said fenugreek hydrocolloid acts as stabilizer, emulsifier, dispersant or rheology modifier.

It is yet another object of the present invention to provide compositions comprising fenugreek hydrocolloid comprising soluble and insoluble dietary fiber to enhance the organoleptic properties and improve the utility and stability of products for food, pharmaceutical or personal care industry.

### SUMMARY OF THE INVENTION

The present invention provides compositions comprising fenugreek hydrocolloid comprising soluble dietary fiber and insoluble dietary fiber. The present invention provides compositions comprising fenugreek hydrocolloid comprising soluble dietary fiber and insoluble dietary fiber for use as healthcare, personal care, food, household care and industrial products wherein fenugreek hydrocolloid acts as a stabilizer, emulsifier, dispersant or rheology modifier

### DETAILED DESCRIPTION OF THE INVENTION

The present invention is directed to compositions comprising fenugreek hydrocolloid comprising soluble dietary fiber and insoluble dietary fiber for use as healthcare, personal care, food, household care and industrial products.

The term “hydrocolloid” includes hydrophilic polymers of vegetable, animal, microbial or synthetic origin that form colloidal solutions or gels with water.

The term “fenugreek hydrocolloid” as used in the present invention refers to dietary fibers from fenugreek comprising soluble dietary fiber and insoluble dietary fiber.

The term “personal care products” as used in the present invention includes, but is not limited to, cosmetics, toiletries, cosmeceuticals for skin, hair, scalp, body and nail care and personal hygiene products for humans and animals.

The term “health care products” as used in the present invention includes, but is not limited to, pharmaceuticals, medical devices, oral care, eye care, ear care or wound care products and the like.

The term “household care products” as used in the present invention includes, but is not limited to, products being employed in a household for surface cleaning, sanitation, laundry cleaning and the like.

The term “industrial products” as used in the present invention includes, but is not limited to, products employed for processing, cleaning and finishing in various industries like textiles, paper, paints, printing, construction, and the like and at places of community health maintenance like hospitals and healthcare centers.

The term “organoleptic” as used in the present invention refers to any sensory property of a product, involving taste, color, odor, texture and mouth feel.

The present invention describes compositions comprising fenugreek hydrocolloid comprising soluble dietary fiber and insoluble dietary fiber. In one embodiment, fenugreek hydrocolloid is obtained from fenugreek seeds. In a further embodiment, fenugreek hydrocolloid of the compositions of the present invention is obtained from fenugreek seeds by an ecofriendly process described in European Patent 1697050B1 (the '050 patent). The process described in the '050 patent yields a combination of dietary fibers, i.e., soluble dietary fibers and insoluble dietary fibers that have synergistic activity when used as hydrocolloids. In one embodiment of the present invention, the ratio of insoluble dietary fiber to soluble dietary fiber in the fenugreek hydrocolloid of the compositions of the present invention is about 0.2 to about 5. In another embodiment of the present invention, the ratio of insoluble dietary fiber to soluble dietary fiber in the fenugreek hydrocolloid of the compositions of the present invention is about 0.5 to about 4. In yet another embodiment of the present invention, the ratio of insoluble dietary fiber to soluble dietary fiber in the fenugreek hydrocolloid of the compositions of the present invention is about 0.8 to about 3. In a further embodiment of the present invention, the ratio of insoluble dietary fiber to soluble dietary fiber in the fenugreek hydrocolloid of the compositions of the present invention is about 1 to about 3. In another embodiment, the fenugreek hydrocolloid of the present invention may have a viscosity greater than 10,000 cps at 2% w/v and protein content of less than 10%.

Compositions of the present invention comprising fenugreek hydrocolloids comprising soluble dietary fiber and insoluble dietary fiber may be used as food and fodder, pharmaceutical, personal care, household care and industrial products. The compositions of the present invention comprising fenugreek hydrocolloids can be used as a healthcare, personal care, food, household care or industrial products, wherein fenugreek hydrocolloids comprising soluble dietary fiber and insoluble dietary fiber is present as an additive.

Fenugreek hydrocolloids can be incorporated in the compositions of the present invention for pharmaceutical, personal care, food industry or the like in a form including, but not limited to, powders, granules or aqueous dispersions. Fenugreek hydrocolloids can be added to healthcare, personal, food, household care or industrial products in an amount ranging from about 0.1% to about 99% by weight of the composition. In one embodiment of the present invention fenugreek hydrocolloid can be added to healthcare, personal care, food, household care or industrial products in an amount ranging from about 0.5% to about 95% by weight of the composition. In another embodiment of the present invention fenugreek hydrocolloid can be added to healthcare, personal, food, household care or industrial products in an amount ranging from about 1% to about 90% by weight of the composition.

Fenugreek hydrocolloids employed in the compositions of the present invention may be in a further modified, functionalized form or may be derivatized with ionic, non-ionic or amphoteric moieties for use in healthcare, personal care, food, household care and industrial products.

In the food industry, especially during the manufacture of ice creams, ice cream shakes and frozen desserts, stabilizers based on gums are generally incorporated to maintain homogeneity, control ice crystal growth during the freezing or aeration process, and to provide slower and more uniform meltdown. Generally, the incorporation of such gum-type stabilizers deleteriously affects the mouth feel of such food products giving a cloying, gummy, sticky or even a greasy feeling. The inventors have surprisingly found that compositions of the present invention comprising fenugreek hydrocolloids, when prepared in the form of a food product, improve the organoleptic properties of these food products, like mouth feel and texture, and function as effective freezing stabilizers as well as emulsifiers. Fenugreek hydrocolloids employed in the compositions of the present invention have a high water holding capacity and are capable of binding quantities of water considerably larger than their weight and retarding the growth of ice crystals when the frozen food is subject to changes in temperature. Thus fenugreek hydrocolloids help maintain smooth texture in ice creams including but not limited to kulfi, gelato, ice cream shakes including but not limited to mastani and frozen desserts including but not limited to frozen custard by slowing down ice crystal growth and restricting flow of ‘free water’ during constant and fluctuating temperatures. Further, the presence of insoluble fibers in fenugreek hydrocolloids employed in the compositions of the present invention reduce the sticky or cloying texture of such food products.

Further, fenugreek hydrocolloids employed in the compositions of the present invention function as stabilizers, emulsifiers and rheology modifiers that provide smooth texture and stability to dairy products including but not limited to yoghurt, gelato, custards, pudding, condensed milk, cream cheese, creamy fruit salad and the like, and maintain their optimum quality for extended time periods. The fenugreek hydrocolloids also help reduce syneresis in dairy products and prevent moisture migration. Fenugreek hydrocolloids of the present invention comprising soluble dietary fiber and insoluble dietary fiber help maintain a smooth and better texture in dairy products. The fenugreek hydrocolloid can increase viscosity as desired when incorporated in food products as thereby function as rheology modifiers. In one embodiment, the fenugreek hydrocolloid of the present invention can generate a viscosity of >10,000 cps at 2% w/v concentration as discussed in the '050 patent. In another embodiment, the fenugreek hydrocolloid of the present invention can generate a viscosity of >50,000 cps at 2% w/v concentration as discussed in the '050 patent.

Further, fenugreek hydrocolloid employed in baked foods provides improved texture, mouth feel, softness, moisture-retention, shelf-life and fatty attributes to them. Fenugreek hydrocolloids in the compositions of the present invention function as a reservoir of bound moisture which is able to slowly release this moisture into the baked food as moisture is passed from the baked food to the ambient atmosphere. The presence of insoluble dietary fiber in the fenugreek hydrocolloid-comprising compositions disrupts the gluey (sticky) texture which would result from the presence of soluble fibers or galactomannans alone. Fenugreek hydrocolloids in the compositions of the present invention can also be used to encapsulate flavors such as cinnamon oil. Further, the lubricity and softness normally provided to baked foods by fatty materials is provided by fenugreek hydrocolloids.

When used in confectionaries, fenugreek hydrocolloids of the compositions of the present invention function as stabilizers by retarding sugar crystallization and as emulsifiers and dispersants by emulsifying and distributing fat particles in the confectionary product. Fenugreek hydrocolloids of the compositions of the present invention also aid fixing flavors in these products.

Fenugreek hydrocolloids of the compositions of the present invention serve as useful and inexpensive emulsifiers, texturizers and film-formers that in the beverage industry function to stabilize flavors and essential oils. The synergistic presence of fenugreek soluble dietary fiber and insoluble fiber provides enhanced emulsification and stabilization properties and also serves as cloud-producing agent and foam-stabilizing agent in the beverage industry.

Fenugreek hydrocolloids can be useful in the production of salad dressings, sauces, gravies, soups, mayonnaise, fillings, puddings or toppings and can be used in pet food and fodder.

Further, gels advantageous in terms of gel strength, break strength, syneresis and heat stability are produced by Fenugreek hydrocolloids employed in the compositions of the present invention and are of benefit in food, fodder, pharmaceutical, personal care, household care and industrial products. Further, fenugreek hydrocolloid may be used as a stabilizer, emulsifier, and carrier or for controlled release of active agents and flavors or fragrances in pharmaceutical, personal care and food industry. Fenugreek hydrocolloids may also help to avoid phase separation in products for pharmaceutical, personal care, food and fodder, household care and industrial products, thereby increasing their stability and shelf-life.

Fenugreek hydrocolloid stabilizes lotions and protective creams. It increases the viscosity, assists in imparting spreading, adds a smooth feel to the skin, and forms a protective coating. Fenugreek hydrocolloid also provides moisture retention or moisturizing benefits to a composition for personal care. It can also serve as a binding agent in the formulation of compact cakes and rouges, and act as an adhesive in the preparation of facial masks and skin treatment compositions. It can also be used as a fixative or styling agent and binder in hair styling products and as a stabilizer and film former in protective creams. In personal care (such as, for example, hair and skin) products, fenugreek hydrocolloids of the compositions of the present invention aid wetting of the skin, scalp or hair, facilitate dirt removal and dissolution, and ease rinsing after application. Fenugreek hydrocolloids in personal care products function, among other things, as rheology modifiers, stabilizers, emulsifiers, binders, dispersants or film formers. Further, the compositions of the present invention comprising fenugreek hydrocolloids may be in the form of oral care products for mouth, gums and teeth such as mouth wash, dentifrice, such as toothpaste, tooth powder, tooth polishes, tooth whiteners, breath fresheners, denture adhesives, and the like.

Topical pharmaceuticals and cosmeceuticals comprising fenugreek hydrocolloids of the present invention as spreading aids, rheology modifiers or film formers include, but are not limited to, skin and hair protective and nourishing sprays, creams, lotions, gels, stick, powder products such as antiseptics, disinfectants, itch relief, sun screens, sun blocks, toning compositions, moisturizers, conditioners, keratolytic or exfoliant compositions, or active skin and hair treatment lotions and creams such as anti-acne, anti-cellulite, anti-aging, skin lightening, anti-dandruff and the like. The Fenugreek hydrocolloids can be used in nail care and hand and foot care compositions such as but not limiting to nail softeners, foot deodorant sprays, moisturizing lotions or anti-cracking creams as well.

Further, non-topical pharmaceuticals comprising fenugreek hydrocolloids of the present invention as binder, rheology modifier, controlled release agent or coating agent include, but are not limited to, tablets, capsules, gel capsules, solutions, suspensions, syrup, enemas, colonics, suppositories, ophthalmic products, ear products, nasal products or other transmucosal products and the like.

In another aspect of the present invention, fenugreek hydrocolloids, comprising soluble dietary fiber and insoluble dietary fiber, as dispersants, rheology modifiers and stabilizers may be employed in the preparation of pharmaceutical and personal care products containing particulates such as, for example, suspensions, emulsions, dermal or oral care products containing microgranules and the like. Fenugreek hydrocolloids can be used as spreading aids and carriers for enhancing the efficacy and delivery of pharmaceuticals and cosmetics and as a vehicle for enhancing the organoleptic properties of the pharmaceutical and cosmetic products.

In a further embodiment, fenugreek hydrocolloids can be employed for tastemasking of, but not limiting to, bitter or non-palatable actives during preparation of pharmaceutical dosage forms. Examples of such actives include, but are not limited to, macrolide antibiotics such as erythromycin, azithromycin and clarithromycin; fluroquinolones such as ciprofloxacin, enrofloxacin, ofloxacin, gatifloxacin, levofloxacin and norfloxacin; cephalosporins such as cefuroxime, cefaclor, cephalexin, cephadroxil and cepfodoxime proxetil; nonsteoroidal, anti-inflammatory and analgesic drugs such as ibuprofen, aspirin, acetaminophen and diclofenac sodium, COX 2 inhibitors such as etaricoxib and celecoxib; antihistamic drugs such as cimetidine, ranitidine, famotidine and chlorpheniramine maleate; oxazolidinones comprising linezolid; and dextromethorphan. The presence of insoluble dietary fibers in fenugreek hydrocolloids of the present invention helps reduce the gummy mouth feel of a composition that has been tastemasked by using fenugreek galactomannan alone.

Further, fenugreek hydrocolloids can be used in compositions for the delivery of active agents that tend to irritate the gastric mucosa, wherein the presence of fenugreek hydrocolloid provides a mucosa-protective benefit.

Fenugreek hydrocolloids may also be used in compositions used for wound care products including, but not limited to, wound dressings, antibiotic creams, ointments, liquid bandages and the like wherein adequate rehydration of the wounded tissue is promoted by fenugreek hydrocolloids. In the case of moist dressings, optimum moisture retention and rehydration is provided by fenugreek hydrocolloids comprising soluble dietary fiber and insoluble dietary fiber than that provided by fenugreek gum or galactomannan alone. Hydrogels based on fenugreek hydrocolloids, comprising soluble dietary fiber and insoluble dietary fiber, may be used for wound healing as well as for medical device implantation or application. The gel strength, biocompatibility, moisture retention abilities and non-toxic nature of these hydrogels make them suitable for such applications.

Fenugreek hydrocolloids comprising soluble dietary fiber and insoluble dietary fiber may be employed for use in household and industrial products such as, for example, detergents, fabric softeners, hard surface cleansers, air treatment gels, those for textile processing, paper and paper making, leather and hide processing, chemical spills containment, printing and paints, building and road construction purposes as rheology modifiers, stabilizers, emulsifiers or dispersants. Fenugreek hydrocolloids also provide optimum lathering and foam stabilization when used in personal care, household care and industrial products.

There is no limitation on the form of the fenugreek hydrocolloid comprising compositions of the present invention, as long as the purpose for which the product is used is achieved.

Further, fenugreek hydrocolloids comprising soluble dietary fiber and insoluble dietary fiber may be combined with other gums or soluble fibers including, but not limited to, cassia, carrageenan, xanthan, agar, guar gum, locust bean gum. Fenugreek hydrocolloids may also be combined with other insoluble dietary fibers including, but not limited to, oat fiber, soy fiber or wheat fiber, and incorporated in the compositions of the present invention.

The amount and type of additional ingredients or excipients in healthcare, personal care, food and industrial products of the present invention varies depending on the product and its function, as is well known to a person skilled in the art.

Exemplary ingredients for food products depending on the final form of the food product include, but are not limited to, acidulants, antioxidants, sequestrants, colors, color retention agent, sweeteners, emulsifiers, fats, oils, flavors, flavor enhancers, flour, flour treatment agents, gums, preservatives, stabilizers, spices, thickeners, bulking agents, vitamins, anticaking agents, antifoaming agents or humectants. Representative examples of each of these include, but are not limited to, acidulants such as citric acid, phosphoric acid, lactic acid, malic acid, tartaric acid, adipic acid, succinic acid, acetic acid, ascorbic acid; antioxidant agent such as glutathione, vitamin C, Melatonin, vitamin E, catalase, superoxide dismutase, peroxidases; sequestrant such as calcium disodium ethylene diamine tetra-acetate, glucono delta-lactone, sodium gluconate, potassium gluconate, sodium tripolyphoshate; food colours such as caramel, annatto, a green dye made from chlorella algae, cochineal, beet juice, turmeric, saffron, paprika; colour retention agents such as ascorbic acid, nicotinic acid; sweeteners such as maple syrup, sugar beet syrup, corn syrup, cane syrup, molasses acesulfame potassium, alitame, aspartame, cyclamate, saccharin, stevioside, sucralose, inulin; emulsifiers such as egg yolk (lecithin); flavours such as diacetyl isoamyl acetate, cinnamic aldehyde, ethyl propionate, limonene, ethyl-(E,Z)-2,4-decadienoate, allyl hexanoate, ethyl maltol, methyl salicylate, benzaldehyde; flavour enhancers such as monosodium glutamate, monopotassium glutamate, calcium diglutamate, monoammonium glutamate, magnesium diglutamate, glutamic acid, guanylic acid, disodium inosinate, maltol, ethyl maltol, glycine and its sodium salt; gums such as gum arabic, gellan gum, guar gum, locust bean gum, xanthan gum; preservatives such as calcium propionate, sodium nitrate, sodium nitrite, sulfur dioxide, sodium bisulfite, potassium hydrogen sulfite, disodium EDTA; stabilizers such as gelatin, carrageenan; spices such as parsley, rosemary, coriander, berberis, mustard, black pepper, garlic, onion, ginger; thickeners such as starches, vegetable gums, and pectin, proteins; bulking agents such as starch; vitamins; anticaking agent such as sodium bicarbonate, sodium ferrocyanide, potassium ferrocyanide, sodium silicate, silicon dioxide, calcium silicate, magnesium trisilicate, talcum powder, sodium aluminosilicate, potassium aluminium silicate, calcium aluminosilicate, bentonite, aluminium silicate, stearic acid, polydimethylsiloxane; anti-foaming agent such as silicone oil; humectants such as glycerine, propylene glycol, glyceryl triacetate, sorbitol, xylitol, maltitol, polydextrose; flours such as wheat flour, corn flour; flour treatment agents such as azodicarbonamide, carbamide, potassium bromate, ascorbic acid, phosphates, malted barley.

The compositions comprising fenugreek hydrocolloids can be prepared by any method known to a person skilled in the art depending on the final form of the product such as healthcare, personal care, food, household care and industrial product.

While the present invention has been described in terms of its specific embodiments, certain modifications and equivalents will be apparent to those skilled in the art and are intended to be included within the scope of the present invention. The following examples merely illustrate the present invention and are not to be construed to limit, in any way, the scope of the invention.

### EXAMPLES

### Example 1

### Comparative Evaluation of Emulsifying Ability of Fenugreek Hydrocolloid of the Present Invention, Fenugreek Soluble Fiber and Microcrystalline Cellulose

The emulsification ability of fenugreek hydrocolloids of the present invention was compared to that of fenugreek soluble fiber that is commercially available from Ceejay Healthcare Private Limited, microcrystalline cellulose, as a representative of insoluble fiber and a physical mix of 1:1 ratio of fenugreek soluble fiber and microcrystalline cellulose.

Oil-in-water emulsions (10 wt % oil) for the study were prepared in the presence of 0.75% w/w of the four emulsifiers discussed above. The dispersions were stirred using homogenizer to obtain emulsions that was stored at 25° C. These emulsions were immediately, after 6 hrs and after 4 days evaluated for particle size distribution by microscopy and for physical appearance.

The initial and after 6 hrs evaluation showed that over 80% of the particles in the emulsion prepared with fenugreek soluble fiber as emulsifier were more than 20μ in size while about 80% of the particles in the emulsion with fenugreek hydrocolloid of the present invention as emulsifier were less than 20μ in size. Even after 4 days, about 80% of the particles in the emulsion prepared with fenugreek hydrocolloids retained their size and were less than 20μ in size while it was not so with emulsions prepared with fenugreek soluble fiber as emulsifier. Emulsion formed with microcrystalline cellulose and a physical mix of 1:1 ratio of fenugreek soluble fiber and microcrystalline cellulose, as emulsifiers, exhibited phase separation before 6 hours.

This shows that fenugreek hydrocolloid of the present invention exhibited better emulsification ability than that exhibited by other emulsifiers evaluated. Further, fenugreek hydrocolloids also produced emulsions with good stability.

### Example 2

### Comparative Evaluation of Water Holding Capacity of Fenugreek Hydrocolloid of the Present Invention, Fenugreek Soluble Fibers and Microcrystalline Cellulose

The water holding capacity of fiber is a measure of the ability of a fiber source to immobilize water within its matrix. The centrifugation and filtration method was used to compare the water holding capacity of fenugreek hydrocolloid employed in the compositions of the present invention, fenugreek soluble fiber with a ratio of insoluble to soluble fibers of 0.04 and microcrystalline cellulose, a representative of insoluble fibers. For the centrifugation method tared centrifuge tubes each containing the specified amounts of dry fiber soaked for 24 h in distilled water were centrifuged at 10000 rpm for 20 min and the supernatant fraction was decanted. The fresh weight of fiber was determined and the water holding capacity was calculated as g water/g fiber. The results obtained as depicted beneath clearly indicate that fenugreek hydrocolloid has a better water holding capacity than the fenugreek soluble fibers commercially available from Ceejay Healthcare Private Limited and microcrystalline cellulose.

| | Water Holding Capacity |
|---|---|
| Sample | gm water/gm fiber |
| Fenugreek soluble fiber | 22.97 |
| Microcrystalline cellulose | <2 |
| Fenugreek hydrocolloid of | 26.375 |
| the present invention | |

### Example 3

### Ice-Cream Containing Fenugreek Hydrocolloid

| | 3A | 3B |
|---|---|---|
| Ingredients | Parts/100 gm | Parts/100 gm |
| Cow's milk | 16.5 | 16.5 |
| Fenugreek hydrocolloid | 4.75 | — |
| Fenugreek soluble fibers | — | 4.75 |
| Sugar | 15.75 | 15.75 |
| Water | 40.0 | 40.0 |
| Fresh cream | 12.5 | 12.5 |
| Mango pulp | 10.5 | 10.5 |

### Procedure:

Sugar was mixed with fenugreek hydrocolloid (in 3A and with fenugreek soluble fibers obtained from Ceejay Healthcare Private Ltd, India in 3B) and blended well. This was added to water slowly under stirring followed by hydration. Cow's milk was boiled and cooled to which fresh cream was added and mixed well. Sugar and fenugreek hydrocolloid in solution were added to milk, followed by addition of fresh mango pulp. Thereafter, the product was put in a refrigerator until it turned into a semisolid. The semisolid mass was stirred in mixer for 10 minutes and again kept in deep freezer to set the ice-cream. Ice cream with fenugreek hydrocolloid (3A) had good mouthfeel, smooth texture and excellent palatability as compared to ice cream with fenugreek soluble fibers only (3B). The ice cream with fenugreek hydrocolloids was free from ice crystals as well.

### Example 4

### Kesari Kaju Kulfi with Fenugreek Hydrocolloid

| Ingredients | Parts/100 gm |
|---|---|
| Cow's milk | 60 |
| Skimmed milk powder | 5.5 |
| Fenugreek hydrocolloid | 1.5 |
| Sugar | 15 |
| Fresh cream | 15 |
| Saffron | 1 |
| Cashews | 2 |

### Procedure:

The milk was heated to boil. The milk powder was added to it and heated till the mixture reduces to about three-fourths by continuous stirring. The mixture was cooled to room temperature and sugar was added followed by bread slices. In a blender, a paste of cashewnuts (kaju) was prepared and this was added to above mixture by blending for 5 to 10 minutes. Kesar (saffron) was added to the blend and mixed. Fresh cream was stirred well in the blender and mixed with above system.

The mixture was poured in the kulfi moulds and put in deep freezer to get delicious kesar kaju kulfi.

### Example 5

### Ice-Cream Lassi (Mastani) with Fenugreek Hydrocolloid

| Ingredients | Parts/1000 gm |
|---|---|
| Milk | 500 |
| Yogurt | 200 |
| Sugar | 120 |
| Fenugreek hydrocolloid | 30 |
| Kesar ice cream | 150 |

### Procedure

The milk was boiled for 15 minutes. In another container small amount of milk and fenugreek hydrocolloid was mixed well and this mixture was poured in boiling milk, the boiling was continued for next 5 minutes. The mixture was allowed to cool to room temperature and kept it in refrigerator for an hour. The yogurt was beaten well and put in the blender to which milk fenugreek hydrocolloid mixture was added along with sugar, ice cubes & blended it well further for 10 minutes. The blended mixture was poured in a glass and ice cream was put on it to get ice cream lassi with fenugreek hydrocolloid.

### Example 6

### Pineapple Pudding with Fenugreek Hydrocolloid

| Ingredients | Parts/500 gm |
|---|---|
| Condensed milk | 180 |
| Fresh cream | 150 |
| Fenugreek hydrocolloid | 50 |
| Pineapple syrup | 100 |
| Pineapple small pieces | 20 |

### Procedure

The fresh cream and the condensed milk were beaten in a blender until mixed well. Fenugreek hydrocolloid was mixed with pineapple syrup and mixed well until the hydrocolloid was completely dissolved. The mixture was allowed to cool at the room temperature. Pineapple pieces were added to the mixture and poured in a bowl and refrigerated for 4 hrs.

### Example 7

### Creamy Fruit Salad with Fenugreek Hydrocolloid

| Ingredients | Parts/1000 gm |
|---|---|
| Cow milk | 350 |
| Fresh cream | 130 |
| Fenugreek hydrocolloid | 50 |
| Fruit pieces (assorted and sweet) | 300 |
| Banana | 60 |
| Sugar | 100 |
| Vanilla Extract | 10 |

### Procedure

Fruit pieces were mixed with slices of bananas in a bowl, covered with plastic wrap and kept in refrigerator. Milk was heated to boil and sugar was added to it. Fenugreek hydrocolloid was mixed with 50 ml of cold milk and added to the hot milk. Vanilla extract was added and the stabilized mixture was allowed to cool down. The fresh cream was beaten well in a mixer to get soft mass. The whipped cream was incorporated into cold stabilized mixture by slow and gentle mixing. The mixture was poured onto the fruit bowl and mixed it very carefully. The resultant recipe was put in dessert bowls and kept in refrigerator for an hour before serving.

### Example 8

### Toothpaste with Fenugreek Hydrocolloid

| Ingredients | % w/w |
|---|---|
| Sorbitol (70%) | 44.30 |
| Fenugreek hydrocolloid | 1.0 |
| Deionized water | 32.0 |
| Calcium carbonate (65% slurry) | 10.0 |
| Sodium monofluorophosphate | 0.75 |
| Sodium saccharin | 0.25 |
| Glycerin | 10.0 |
| Sodium lauryl sulphate | 0.40 |
| Methyl paraben | 0.10 |
| Peppermint Flavor | 1.2 |
| Total | 100 |

### Procedure:

Part of fenugreek hydrocolloid was dispersed into 75% of deionized water and mixed. Remaining part of fenugreek hydrocolloid was dispersed into sorbitol using a low shear mixer until smooth. Both the parts were mixed together and calcium carbonate slurry was added to the fenugreek hydrocolloid system and mixed well. Sodium monofluorophosphate and sodium saccharin were dissolved in a portion of remaining deionized water and then added to glycerin. The glycerin mixture was then added to the mixer. Sodium lauryl sulphate and methyl paraben was added to remaining portion of deionized water and dissolved. The solution was added to above mixture and blended. The flavor was added with further mixing and the resultant paste was filled in collapsible plastic tube.

### Example 9

### Moisturizing Lotion with Fenugreek Hydrocolloid

| Ingredients | % w/v |
|---|---|
| Carbopol 1342 | 3.15 |
| Triethanolamine | 1.50 |
| Disodium EDTA | 0.10 |
| Sorbitol | 3.50 |
| Aloe vera gel | 15.0 |
| Fenugreek hydrocolloid | 1.5 |
| Tricapryl citrate | 3.0 |
| Coconut oil | 3.0 |
| Cocoa butter | 0.04 |
| Stearic acid | 5.5 |
| Cetyl alcohol | 3.0 |
| PEG-75 lanolin | 1.0 |
| Dimethicone | 1.0 |
| Propylparaben | 0.1 |
| Methylparaben | 0.20 |
| Deionized water to make | 100 ml |

### Procedure:

Fenugreek hydrocolloid was dispersed in water and mixed well; to which all water soluble ingredients were added to make an aqueous phase. Oily ingredients like coconut oil, stearic acid and rest of oil soluble ingredients were blended and added to the above aqueous phase containing fenugreek hydrocolloid. Oil in water system was homogenized for 10 minutes and fragrance was added towards the end to get a moisturizing lotion.

### Example 10

### Tile Cleanser with Fenugreek Hydrocolloid

| Ingredients | % w/w |
|---|---|
| Deionized water | 40.0 |
| Fenugreek hydrocolloid | 0.5 |
| Magnesium aluminium silicate | 1.5 |
| Calcium carbonate (65% slurry) | 46.0 |
| Benzyl alkyl sulphonic acid | 12.0 |
| Sodium hydroxide (50 %) | Adjust pH 8 to 9 |
| Total | 100 |

### Procedure:

Fenugreek hydrocolloid is dispersed in water and other ingredients were added in the dispersion in the order as shown in the table. The pH of the suspension was adjusted using sodium hydroxide solution to 8 to 9 to get the cleanser solution with fenugreek hydrocolloid.

What is claimed is:

1. A composition comprising fenugreek hydrocolloid wherein said fenugreek hydrocolloid comprises soluble dietary fiber and insoluble dietary fiber.

2. The composition of claim 1 wherein said fenugreek hydrocolloid is present in an amount of about 0.1% to about 99% by weight of said composition.

3. The composition of claim 1 wherein said soluble dietary fiber is present in an amount of about 5% to about 95% by weight of said fenugreek hydrocolloid.

4. The composition of claim 1 wherein said insoluble dietary fiber is present in an amount of about 5% to about 95% by weight of said fenugreek hydrocolloid.

5. The composition of claim 1 wherein the ratio of said insoluble dietary fiber to soluble dietary fiber is about 0.2 to about 5.

6. The composition of claim 5 wherein the ratio of said insoluble dietary fiber to soluble dietary fiber is about 0.8 to about 3.

7. The composition of claim 6 wherein the ratio of said insoluble dietary fiber to soluble dietary fiber is about 1 to about 3.

8. The composition of claim 1 wherein said composition is a healthcare, personal care, food, household care or industrial product.

9. The composition of claim 1 for use as a healthcare, personal care, food, household care or industrial product wherein said fenugreek hydrocolloid is an additive.

10. The composition of claim 9 wherein said composition is an ice cream, ice cream shake, frozen dessert, yoghurt, gelato, custards, condensed milk, cream cheese, bread, confectionery, beverage, salad dressing, sauce, gravy, soup, mayonnaise, pudding, topping, pet food, lotion, skin cream, compact cake, rouge, shampoo, toothpaste, tooth powder, breath freshener, sunscreen, moisturizer, conditioner, nail softener, tablet, capsule, suspension syrup, wound dressing, ointment, detergent, fabric softener or surface cleaner.

11. The composition of claim 1 for use as a healthcare, personal care, food, household care or industrial product wherein said fenugreek hydrocolloid acts as a stabilizer, emulsifier, dispersant or rheology modifier.

12. A method of preparing a preparation of a healthcare, personal care, food, household care or industrial product comprising incorporating fenugreek hydrocolloid as claimed in claim 1 in the preparation.

13. A method of preparing a preparation of a healthcare, personal care, food, household care or industrial product exhibiting enhanced organoleptic properties comprising incorporating fenugreek hydrocolloid as claimed in claim 1 in the preparation.

14. A method of preparing a preparation of a healthcare, personal care, food, household care or industrial product exhibiting improved stability comprising incorporating fenugreek hydrocolloid as claimed in claim 1 in the preparation.
